(19) United States
(12) Patent Application Publication
(10) Pub. No.: US 2015/0223725 A1
(43) Pub. Date: Aug. 13, 2015

(54) MOBILE MANEUVERABLE DEVICE FOR WORKING ON OR OBSERVING A BODY

(71) Applicants: FRAUNHOFER-GESELLSCHAFT ZUR FOERDERUNG DER ANGEWANDTEN FORSCHUNG E.V., Munich (DE); CHARITE-UNIVERSITAETSMEDIZIN BERLIN, Berlin (DE)

(72) Inventors: Sebastian Engel, Muenster (DE); Erwin Keeve, Potsdam (DE); Christian Winne, Berlin (DE); Eckart Uhlmann, Kiebitzreihe (DE)

(21) Appl. No.: 14/411,602

(22) PCT Filed: Jun. 28, 2013
(86) PCT No.: PCT/EP2013/063699

§ 371 Date: Dec. 29, 2014

(30) Foreign Application Priority Data
Jun. 29, 2012 (DE) 10 2012 211 378.9
Nov. 5, 2012 (DE) 10 2012 220 116.5

(51) Int. Cl. A61B 5/06; A61B 17/00; A61B 19/00; A61B 5/00; A61B 1/05; A61B 1/00

(52) U.S. Cl. CPC A61B 5/066; A61B 1/05; A61B 1/00009; A61B 19/5244; A61B 1/00006; A61B 5/725; A61B 19/54; A61B 1/00163; A61B 1/00011; A61B 17/00234; A61B 2019/5236; A61B 2019/524; A61B 2019/5248; A61B 2560/0223; A61B 2019/5454; A61B 2019/5466; A61B 2019/5265; A61B 2019/5287; A61B 2019/5261

(57) ABSTRACT

The invention relates to a mobile, maneuverable device (1000) having a mobile device head (100), particularly a medical, mobile device head (100) having a distal end for the purpose of arrangement relative to a body, particularly insertion or attachment on the body, having at least one mobile device head (100) designed for the purpose of manual or automatic guidance, having a guide device (400) designed for the purpose of navigation, having an image data processing device (430) which compiles a map (470) of the environment by means of the image data, and having a navigation device which can indicate at least one position (480) of the device head (100) using the map, by means of the image data and an image data stream.

The invention relates to a mobile, maneuverable device such as a tool, an instrument, or a sensor or the like, particularly for working on or observing a body. The invention preferably relates to a mobile maneuverable medical device, particularly for working on or observing a biological body, particularly tissue. The invention preferably relates to a mobile maneuverable non-medical device, particularly for working on or observing a technical body, particularly an object. The invention also relates to a method for maneuvering—particularly calibrating—the device, particularly in the medical or non-medical field.

A mobile maneuverable device named above can particularly be a tool, instrument, or sensor, or a similar device. In particular, a mobile maneuverable device—preferably a medical or non-medical device—named above can be an endoscope, a pointer instrument, or an instrument or tool—preferably a non-medical instrument or tool or a medical instrument or tool, particularly a surgical instrument or tool. The mobile maneuverable device has at least one mobile device head designed for the purpose of manual or automatic guidance, and a guide device which is designed for the purpose of navigation, in order to enable an automatic guidance of the mobile device head.

In robotics, particularly in the medical or non-medical field, approaches have been developed for a mobile maneuverable device of the type named above. At this time, an approach is followed for incorporating a guide device which uses endoscopic navigation and/or instrument navigation, wherein optical or electromagnetic tracking methods are used for the navigation. By way of example, modular systems are known for an endoscope having system modules which expand the same, such as a tracking camera, a computer, and a visual display device, for displaying a clinical navigation.

Tracking fundamentally means a method for creating a path and/or tracing, which serves the purpose of following moved objects—in the present case particularly the mobile device head. The aim of this following is usually the depiction of the observed, actual movement, particularly relative to a mapped environment, for a technical use. The latter can be the meeting of the tracked (guided) object—particularly the mobile device head—with another object (e.g. a target point or a target trajectory in the environment), or simply the knowledge of the momentary “pose”—that is, the position and/or orientation—and/or movement state of the tracked object.

To date, absolute data relating to the position and/or orientation (pose) of the object and/or the movement of the object is generally used, for example in the system named above. The quality of the determined pose and/or movement information firstly depends on the quality of the observation, the tracking algorithm used, and the modeling process which serves the purpose of compensating unavoidable measurement error. Without modeling, the quality of the determined position and movement information is generally comparably poor, however. At present, absolute coordinates or a mobile device head—for example in a medical application—are inferred, by way of example, from the relative relationship between a patient tracker and a tracker for the device head. In such modular systems, termed absolute tracking modules, the additional complexity—in time and space commitments—for the portrayal of the required trackers is fundamentally problematic. The space requirement is enormous, and is extremely problematic in an operation room with a number of personnel.

As such, moreover, there must be adequate navigation information available. This means that, in tracking methods, a signal connection must generally be maintained between trackers and an image data capture device—for example to a tracking camera. This can be an optical or electromagnetic signal connection or the like, by way of example. If such a signal connection—particularly an optical connection—is broken, for example when personnel move into the image capture line between the tracking camera and a patient tracker, the necessary navigation information is missing and the guidance of the mobile device head must be interrupted. In the case of an optical signal connection in particular, this problem is known as the so-called “line of sight” problem.

A more stable signal connection can be created by means of an electromagnetic tracking method, by way of example, which is less susceptible than an optical signal connection. However, such electromagnetic tracking methods are necessarily less precise and more sensitive to electrical or ferromagnetically conductive objects in the measurement space. This is particularly relevant in the case of medical applications because the mobile, maneuverable device is intended to regularly support surgical operations or the like, and the presence of electrical of ferromagnetically conductive objects in the measurement space—that is, in the operating room—can be the norm. A mobile, maneuverable device which largely avoids the problems arising in the classical tracking sensor system used for navigation, as described above, is desirable. This particularly concerns the problems of optical or electromagnetic tracking methods as named above. However, the precision of a guide device used for navigation should be as great as possible in order to enable the most precise possible robotics application nearer to the mobile maneuverable device—particularly a medical application of the mobile, maneuverable device.

Moreover, however, there is also the problem that the stability of a stationary position of a patient tracker or locator is significant for the precision of the tracking when the patient data is registered. In practice, in an operating room with a number of personnel, this can likewise not always be assured. In principle, a mobile maneuverable device, having a tracking system, which is improved in this respect is known from WO 2006/131373 A2, wherein the device is advantageously designed for determining and measuring a position in space and/or an orientation in space of bodies, without contact.

New approaches, particularly in the medical field, attempt to support the navigation of a mobile device head by means of intraoperative magnetic resonance tomography, or computer tomography in general, by coupling said device head to an imaging device. The recording, by way of example of image data obtained by means of endoscopic video data, using a preoperative CT capture, is described in the article by Mirota et al.: “A System for Video-Based Navigation for Endoscopic Endonasal Skull Base Surgery,” IEEE Transactions on Medical Imaging, Vol. 31, No. 4, April 2012, or in the article by Burschka et al.: “Scale-invariant registration of monocular endoscopic images to CT-scans for sinus surgery,” in Medical Image Analysis 9 (2005) 413-426. An essential aim of the recording of image data, the same obtained by way of example by means of endoscopic video data, is an improvement in the precision of the recording.

Such approaches, on the other hand, are comparably inflexible, however, because it is always necessary to prepare a second image data source—for example in a preoperative CT scan. In addition, CT data are associated with great effort and high costs. The acute and flexible availability of such approaches at any given, desired point in time—for example spontaneously during an operation—is therefore not possible, or is only possible to a limited degree and with preparation.

The newest approaches forecast the possibility of using methods for simultaneous localization and mapping in vivo for the purpose of navigation. A fundamental study of this has been described in, by way of example, the article by Mountney et al. for the 31st Annual International Conference of the IEEE EMBS Minneapolis, Minn., USA, Sep. 2-6, 2009 (978-1-4244-3296-7/09). In the article by Grasa et al.: “EKF monocular SLAM with relocalization for laparoscopic sequences,” in 2011 IEEE International Conference on Robotics and Automation, Shanghai, May 9-13, 2011 (978-1-61284-385-8/11), a real-time application is described at 30 Hz for a 3D model within the framework of a visual SLAM with an extended Kalman filter (EKF). The pose (position and/or orientation) of an image data capture device is taken into account in a three-point algorithm. Real-time usability and robustness with respect to a moderate level of object movement have been tested.

These approaches fundamentally promise success, but nevertheless can still be improved.

The invention proceeds from this point, addressing the problem of providing a mobile maneuverable device and a method which enable a navigation in an improved manner, and nonetheless allow improved precision for the guidance of a mobile device head. The problem addressed is particularly that of providing a device and a method in which navigation is possible with comparably little complexity and with increased flexibility, particularly in situ.

In particular, it should be possible to automatically guide a non-medical, mobile device head having a distal end into an arrangement relative to a technical body, particularly an object, particularly having a distal end for the purpose of the insertion or attachment on the body. In particular, the invention aims to provide a non-medical method for the maneuvering, and particularly calibration, of the device.

In particular, it should be possible to automatically guide a medical, mobile device head having a distal end into an arrangement relative to a biological body, particularly a tissue-like body, particularly having a distal end for the purpose of insertion or attachment on the body. In particular, the invention aims to provide a medical method for the maneuvering, and particularly calibration, of the device.

The problem with respect to the device is addressed by the invention by means of a device according to claim 1 having a mobile device head. The device is preferably a mobile maneuverable device such as a tool, instrument, or sensor or the like, particularly for the purpose of working on or observing a body.

The device is particularly a medical, mobile device having a medical, mobile device head, such as an endoscope, a pointing instrument, or a surgical instrument or the like, having a distal end for the purpose of being arranged relative to a body, particularly body tissue, preferably for insertion or attachment on the body, and particularly on a body tissue, particularly for the purpose of working on or observing a biological body such as a tissue-like body or similar body tissue.

The device is particularly a non-medical, mobile device having a non-medical, mobile device head, such as an endoscope, a pointing instrument, or a tool or the like, having a distal end for the purpose of being arranged relative to a body, particularly a technical object such as a device or an apparatus, preferably for insertion or attachment on the body, particularly on an object, and particularly for the purpose of working on or observing a technical body such as an object or device or a similar apparatus.

The term “distal end of the device head” means an end of the device head which is distant from a guide device, particularly an end of the device head which is the furthest away. Accordingly, a “proximal end” of the device head means an end of the device head positioned near to a guide device, particularly on the end which is closest to the device head.

According to the invention, the device has:
at least one mobile device head designed for the purpose of manual or automatic guidance,
a guide device, wherein the guide device is designed for the purpose of providing navigation information for the guidance of the mobile device head, wherein the distal end thereof can be guided in the near environment (NU),
an image data capture device which is designed to detect and provide image data of an environment (U) of the device head—particularly continuously,
an image data processing device which is designed to compile a map of the environment (U) by means of the image data,
a navigation device which is designed to provide at least one position of the device head in the near environment (NU) using the map, by means of the image data and an image data stream, in such a manner that the mobile device head can be guided using the map.

In addition, a guiding means is included according to the invention which has a position reference with respect to the device head, and is functionally assigned to the same, wherein the guiding means is designed to give details on the position of the device head in the map with respect to the environment (U), wherein the environment (U) goes beyond the near environment (NU).

The position reference of the guiding means with respect to the device head can advantageously be stationary. However, the position reference need not be stationary as long as the position reference can be changed or moved in a manner permitting determination thereof, or in any case can be calibrated. This can be the case, by way of example, if the device head is attached on the distal end of a robot arm as part of a maneuvering apparatus, and the guiding means is attached on the robot arm. The variance in the position reference between the guiding means and the device head, said position reference being not stationary but fundamentally deterministic, and said variance produced by errors or expansions, for example, can be calibrated in this case.

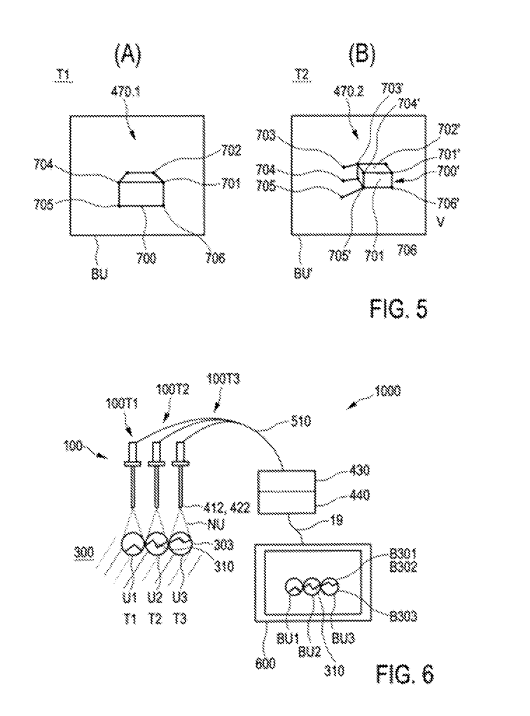

The term “image data stream” means the stream of image data points over time, created when a number of image data points are observed at a first and a second time point while the position, direction, and/or speed of the same is/are varied for a defined passage surface. One example is explained in FIG. 5.

The guiding means preferably, but not necessarily, comprises the image data capture device. By way of example, in the case that the device head is a simple pointer instrument with no optical sight, the guiding means advantageously has a separate guide lens. The guiding means preferably has at least one lens, particularly a target and/or guide lens and/or an external lens.

The guiding means can also additionally or alternatively comprise a further orientation module—for example a movement module and/or an acceleration sensor or a similar system of sensors, designed to provide further detail on the position, and particularly the pose (position and/or orientation), and/or the movement of the device head with respect to the map.

A movement module, particularly in the form of a movement sensor system, such as an acceleration sensor, a speed sensor, a gyroscopic sensor, or the like, is advantageously designed to provide further detail on the pose [position and/or orientation] and/or the movement of the device head with respect to the map.

It is further advantageous that at least one, and optionally multiple mobile device heads can be guided with reference to the map.

The term “navigation” fundamentally means any type of map compiling which specifies a position in the map and/or provides a target point in the map, advantageously in relation to the position: in a wider sense, that is, the determination of a position with respect to a coordinate system and/or the provision of a target point, particularly the provision of a route between the position and the target point which can be advantageously seen on the map.

The invention also leads to a method according to claim 30, particularly for the maneuvering, and particularly calibration, of a device having a mobile device head.

The invention proceeds from a cartographic process and navigation in a map, based substantially on image data, for the environment of the device head in the wider sense—that is, an environment which is not bound to a near environment of the distal end of the device head, such as the visually detectable near environment on the distal end of an endoscope. The method can be carried out with a non-medical, mobile device head having a distal end for the purpose of arrangement relative to a technical body, or with a medical, mobile device head having a distal end for the purpose of arrangement relative to a tissue-like body, particularly with a distal end for the purpose of insertion or attachment on the body.

The method is particularly suitable in one implementation simply for calibration of a device having a mobile device head.

The concept of the invention is the possibility, by means of the guiding means, of mapping an environment from another perspective of the distal end of the device head—for example from the proximal end thereof—such as from the perspective of a proximal end of the device head. This could be, by way of example, the perspective of a guide lens of an external camera, attached on the handle of an endoscope. Because there is a position reference with respect to the device head for the guiding means, a mapping of the device head and a navigation with respect to such a map of the environment can still allow a reliable guidance of the distal end of the device head in the near environment of the same.

The environment (by way of example, in the medical field, the surface of a face, or in the non-medical field, a motor vehicle body, for example) can be disjunct from the near environment (e.g., the interior space of a nose, or in the non-medical field, by way of example, an engine compartment). In particular, in this case the device and/or method is non-invasive—that is, with no physical interaction with the body.

At the same time, such an environment can also include a near environment. By way of example, a near environment can include an operation region in which a lesion is treated, wherein a distal end of the endoscope is guided in the near environment by means of a navigation in a map which has been compiled in an environment adjacent to the near environment. In this case as well, the device and/or a method is non-invasive to the greatest possible degree—that is, with no physical interaction with the body—particularly if the environment does not include an operation environment of the distal end of the mobile device head.

The near environment can be an operation environment of the distal end of the mobile device head, and the near environment can include the specific image data which is detected in the visual range of a first lens of the image data capture device on the distal end of the mobile device head.

In the case where the near environment is potentially immediately adjacent to the environment, this approach can be used synergistically to collect image data from the near environment, and an approximate expansion of the same, and simultaneously map the entire environment. As such, the environment can include a region which is in the near environment and beyond the operation environment of the distal end of the mobile device head.

First, the special advantage results that, put briefly, it is possible to largely avoid complex and inflexible classical tracking sensors.

Moreover, the concept allows the possibility of increasing the precision of the map by means of an additional guiding means—e.g. a movement module or a lens or a similar orientation module. According to the concept of the invention, this creates the prerequisite that the at least one mobile device head can only be guided using the map. In particular, according to the concept [of the invention], the image data itself is used to compile a map—that is, enables a purely image data-based mapping and navigation of a surface of a body as a result. This can refer both to outer and inner surfaces of a body. Particularly in the medical field, by way of example, surfaces of eyes, noses, ears, or teeth can be used for the patient registration. The approach of using an environment which is disjunct from the near environment for the purpose of mapping and navigation also has the advantage that the environment has sufficient reference points which can serve as markers and which can be more precisely detected. In contrast, the properties can be used for capturing image data of a near environment, particularly an operation environment, for improved imaging of the lesion.

The invention can be used in a medical field and in a non-medical field equally as well, particularly non-invasively and without physical intervention on a body.

The method can preferably be limited to a non-medical field.

The invention is preferably, particularly within the scope of the device, not limited to an application in the medical field. Rather, it can very much be used in a non-medical field as well. The concept presented above can be used in a particularly advantageous manner in the assembly or maintenance of technical objects such as motor vehicles or electronics. By way of example, tools can be equipped with the system presented above, and navigated via the same. The system can increase the precision in assembly tasks performed by industrial robots, and/or make it possible to realize assembly tasks which were previously not possible using robots. In addition, the assembly task of a worker/mechanic can be simplified—for example by instructions of a data processor fixed to the tool—based on the concept presented above. By way of example, by adding monitoring, it is possible to reduce the extent of work by adding support, and/or increase the quality of the executed task as a result of the use of this navigation option in connection with an assembly tool (for example, a cordless screwdriver) in a construction process (e.g. a motor vehicle body), or an assembly (e.g. a bolted connection for spark plugs) of a component (e.g. spark plugs or bolts), by means of a data processing.

The device and a method are preferably capable of performing in real-time, particularly with the continuous provision and real-time processing of the image data.

In the scope of one particularly preferred implementation, the navigation is based on a SLAM method, particularly a 6D SLAM method, and preferably a SLAM method combined with a KF (Kalman filter), particularly preferably a 6D SLAM method combined with an EKF (extended Kalman filter). By way of example, video images of a camera, or a similar image data capture device, are used for the purpose of compiling a map. The device head is navigated and guided using the map, particularly exclusively using the map. It has been shown that the further movement sensor system used to increase the precision is sufficient for achieving a significant improvement in precision, particularly into the sub-millimeter region.

The invention is based on the recognition that a fundamental problem of the purely image data-based navigation and guidance using the map is that the precision of approaches based on image data to date depends on the resolution of the lens used in the image data capture device for the navigation and guidance of the device head. The demands of real-time capability, precision, and flexibility are potentially in conflict. The invention is based on the recognition that these demands can all still be met satisfactorily and harmoniously when a guiding means is used which is designed to provide further details on the pose and/or movement of the device head with respect to the map.

The invention is based on the recognition that a fundamental problem of the purely image data-based navigation and guidance using a map is that the precision of approaches based on image data to date depends on the number of the image data capturing units and the scope of the simultaneously detected environment regions, for the navigation and guidance of the device head. Further guiding means, such as movement modules, by way of example, such as a system of sensors for measuring acceleration, such as acceleration sensors or gyroscopes, for example, are equally capable of further increasing the precision, particularly with respect to a map of the environment—including the near environment—which is particularly suitable for the purpose of instrument navigation.

To the extent that the concept of the invention based upon [sic] enabling a navigation and guidance only using the map, this means that the guide device can have an absolute tracking module—for example initially, or in special situations—particularly a system of sensors or the like, which can be activated with limited functionality temporarily for the purpose of compiling the map of the near environment, and is deactivated most of the time. This does not contradict the concept of only guiding a mobile device head by means of the map, because, in contrast to methods known to date, it is possible for an absolute tracking module with an optical or electromagnetic basis to not be constantly activated, in order to enable a sufficient navigation and guidance of the device head.

Advantageous implementations of the invention are found in the dependent claims, and indicate details of advantageous possibilities for realizing the concept explained above within the scope of the problem addressed thereby, and with respect to further advantages.

In the scope of one particularly preferred implementation of the invention, the mobile maneuverable device further comprises a control and maneuvering apparatus which is designed for the purpose of guiding the mobile maneuverable device, using the map, according to a pose and/or movement of the device head. As such, it is particularly preferred that the maneuvering apparatus can be designed for the purpose of automatically guiding the mobile device head via a control connection, by means of the control, and the control is preferably designed for the purpose of navigating the device head via a data coupling, by means of the guide device. By way of example, in this manner, it is possible to provide a suitable control loop, wherein the control connection thereof is designed for the purpose of transmitting a TARGET pose and/or a TARGET movement of the device head, and the data coupling is designed for the purpose of transmitting a CURRENT pose and/or a CURRENT movement of the device head. It is fundamentally possible to use the map data so obtained in the navigation of the instrument, or for the purpose of matching with further image data, such as CT data or MRT data, for example, due to the increased precision of the map and navigation, as well as the guidance.

It is particularly preferred that the image data capture device has at least a number of lenses which is [sic: are] designed for the purpose of detecting image data of a near environment. The number of lenses can include a single lens, or two, three, or more lenses. In particular, a monocular or binocular principle can be used. The image data capture device overall can fundamentally be designed in the form of a camera, particularly as part of a camera system having a number of cameras. By way of example, in the case of an endoscope, a camera installed in the endoscope has proven advantageous. In general, the image data capture device can have a target sighting lens which sits on a distal end of the device head, wherein the sighting lens is designed for the purpose of capturing image data of a near environment on a distal end of the device head, particularly as a sighting lens installed in the device head.

In particular, a camera or another type of guide lens can sit on another position of the device head, by way of example on a shaft, and particularly a shaft of an endoscope. In general, the image data capture device can have a guide lens which sits at a guide position at a distance from a distal end, particularly at a proximal end of the device head and/or on the guide device. In this case, the guide lens is advantageously designed for the purpose of capturing the image data of a near environment of a guide position; that is, an environment which is disjunct from the near environment on a distal end of the device head. Because the region of the image data used for the navigation is fundamentally insignificant, the guide lens can fundamentally be mounted at any suitable point of the device head and/or tool, instrument, or sensor or the like, such that the movement of the device head—by way of example an endoscope—and the assignment of the position is [sic] still possible, or is more precise.

The system is also functional if the camera never penetrates a body.

A multitude of cameras and/or lenses can fundamentally be included, all of which access the same map. However, it can also be contemplated that different maps are compiled, for example if different sensors, such as ultrasound, radar, and cameras are used, and these are functionally assigned and/or registered to different maps continuously by shape, profile, etc.

As such, the invention fundamentally provides a guide device, having an image data capture device, with greater precision if multiple cameras or lenses are operated at the same time on a device head or a moving part of the automatic guidance [sic]. In particular, this leads in general to an implementation wherein a first lens advantageously captures image data and a second lens advantageously captures second image data which is spatially offset. In particular, the first and second image data are captured at the same time. The precision of the localization and map compiling can be increased by further lenses—for example by two or more lenses. By using different imaging units—for example 2D optical image data with radar data—this precision can be additionally increased.

In one variant, the same lens captures first image data and second image data, particularly first and second spatially identical image data, which are offset in time. Such an implementation is particularly suitable in combination with a further advanced image data processing device. The further advanced image data processing device advantageously has a module which is designed to recognize target movements, and to incorporate these into the compiling of a map of the near environment. The target movements are advantageously target body movements which can advantageously be detected according to a physiologic pattern—by way of example rhythmic target body movements such as respiration movements, a heartbeat movement, or a tremor movement.

If more than one lens captures different environments, or partially different environments, it is possible for movement to be detected on the basis of comparing the different environment data. In this case, the moving regions are separated from the fixed regions, and the movement is calculated and/or estimated.

It is particularly preferred that a pose (that is, position and/or orientation) and/or movement of the device head can be indicated using the map, relative to a reference point on an object in an environment of the device head. A guide device advantageously has a module for the purpose of marking a reference point on the object such that the same can be used in a particularly advantageous manner for navigation. The reference point is particularly preferably a part of the map of the near environment—that is, the near environment in the target region, such as on the distal end of an endoscope or a distal end of a tool or sensor, by way of example.

However, the region of the navigation and/or the image data used for the navigation is basically not significant. The movement of the device head and the assignment of the position can still occur, or can occur more precisely with respect to other environments of the device head. In particular, the reference point can be outside of the map of the near environment and serve as a marker. Preferably, it is possible to indicate a certain relation between the reference point and a map position. In this way, the device head can still be navigated, due to the fixed relationship, even if a guide lens provides image data of a near environment which does not lie [in] a work space under an endoscope, a microscope, or a surgical instrument or the like. By adding certain objects, e.g. printed surfaces, to the environment, the system can work more precisely with regard to the localization and map compiling.

It is particularly preferred that the image data processing device is designed to identify a reference point on an object on a visual image with a fixed position of an auxiliary image following a predetermined test. The overlap of the map with external images as a part of a known matching, marking, or registering method particularly serves the purpose of registering the patient in medical applications. It has been found that a more reliable registration can be made due to the concept explained above as part of the present implementation.

In particular, a visual image can be recorded and/or complemented with an auxiliary image. This does not happen continuously, nor in a manner which is similarly essential for carrying out the method. Rather, it is an initial measure, or a measure which is available in regular intervals, as an assistance. A continuous updating process can also be contemplated depending on the available computing power.

A visual image based on the map compiled according to the concept according to the invention has been shown to be of high quality in the identification or registering of high-resolution auxiliary images. An auxiliary image can particularly be a CT or MRT image.

One implementation advantageously leads to a method for the visual navigation of an instrument, having the steps:
mapping of the environment for the purpose of compiling a land map, particularly compiling external and internal surfaces of the environment,
simultaneous localization of an object in the environment—at least for the purpose of determining a position and/or orientation (POSE) of the object in the environment, particularly using a SLAM method—
   by means of an image data capture device such as a capture unit, particularly a 2D or 3D camera or the like used for an imaging data capture of the environment, and
   by means of a navigation device and a movement module for the purpose of movement navigation in the environment, particularly for distance and speed measurement.

A guide device is particularly designed to particularly precisely generate a localization of the object from the data capture of the environment, wherein the processing of the data capture from the capture unit can occur in real time. In this way, it is possible to guide the at least one mobile device head essentially in situ using the map, without additional assistance.

The concept, or one of the implementations, has proven itself advantageous in a number of technical application areas, such as robotics, for example—particularly in medical technology or in a non-medical field. As such, the subject matter of the claims particularly comprises a mobile maneuverable medical device and a particularly non-invasive method for working on or observing a biological body such as a tissue or the like. This can particularly be an endoscope, a pointer instrument, or a surgical instrument or similar medical device for the purpose of working on or observing a body, or for the purpose of detecting its own position, and/or the instrument position, relative to the environment.

As such, the subject matter of the claims particularly comprises a mobile, maneuverable, non-medical device and a particularly non-invasive method for working on or observing a technical body, such as an object or a device or the like. By way of example, the concept can be used successfully in industrial work, positioning, or monitoring processes. However, for other applications as well, in which a claimed mobile maneuverable device—for example as part of an instrument, tool, or sensor-like system—is used according to the described principle, the concept as described, relating substantially to image data, is advantageous. In summary, these applications include a device wherein a movement of a device head is detected by means of image data and a map is compiled with the support of a movement sensor system. This map alone is used according to the concept primarily for navigation. If multiple device heads, such as instruments, tools, or sensors, and particularly an endoscope, a pointer instrument, or a surgical instruments [sic], are used, each having at least one mounted imaging camera, it is then possible that all of these access and/or update the same image map for the purpose of navigation.

Exemplary embodiments of the invention are described below with reference to the drawings in comparison to the prior art, which is likewise illustrated in part—and this in medical application settings wherein the concept is implemented with respect to a biological body. Nevertheless, the embodiments also apply for a non-medical application setting, wherein the concept is implemented with respect to a technical body.

The drawings do not necessarily illustrate the exemplary embodiments to scale. Rather, the drawings are, where it serves the purpose of better understanding, presented in schematic and/or slightly distorted form. As regards expansions of the teaching which can be directly recognized in the drawings, reference is hereby made to the relevant prior art. In this case, it must be noted that numerous modifications and adaptations can be made with respect to the shape and the details of an embodiment without departing from the general idea of the invention. The features of the invention disclosed in the description, in the drawings, and in the claims can be essential for the implementation of the invention individually or in any arbitrary combination. In addition, all combinations of at least two features disclosed in the description, in the drawings, and/or in the claims fall within the scope of the invention. The general idea of the invention is not limited to the exact form or the details of the preferred embodiments shown and described below, nor to a subject matter which would be limited in comparison to the subject matter claimed in the claims. Where measurement ranges are indicated, all values lying within the named boundaries are hereby disclosed as boundary values, and can be used and claimed in any and all manners. Additional advantages, features, and details of the invention are found in the following description of the preferred embodiments, as well as in reference to the drawing, wherein:

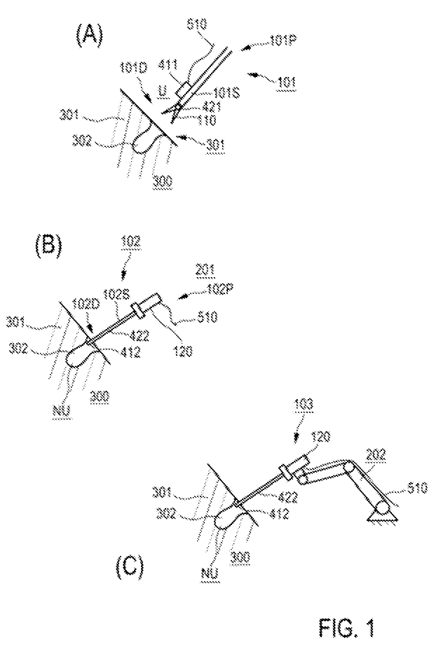

FIG. 1 shows exemplary embodiments of mobile maneuverable devices in a relative position to a body surface—in view (A) with a device head in the form of a gripping instrument, in view (B) with a device head in the form of a hand-guided instrument, such as an endoscope, for example, and in view (C) in the form of a robot-guided instrument such as an endoscope or the like;

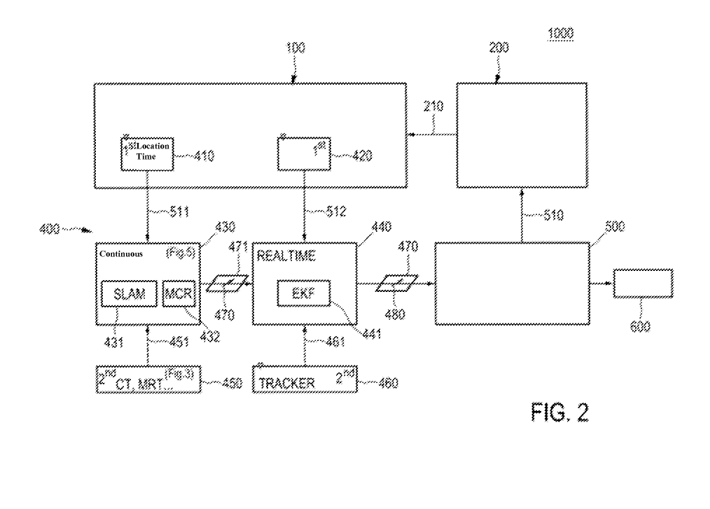

FIG. 2 shows a general schema for the purpose of illustrating a fundamental system and the functional components of a mobile maneuverable device according to the concept of the invention;

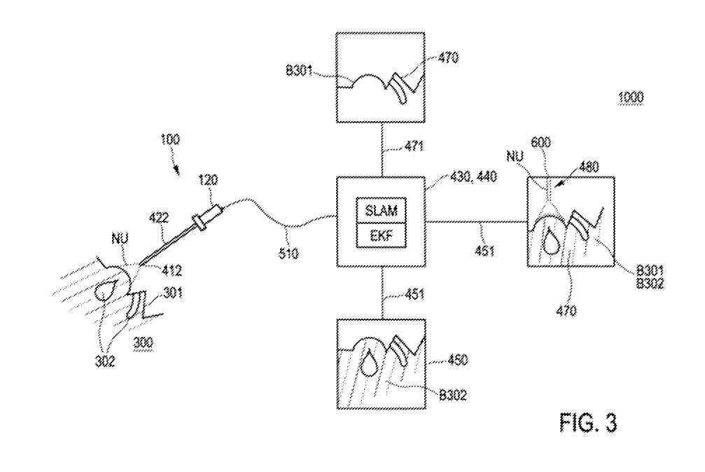

FIG. 3 shows a basic concept using the mobile maneuverable device for the purpose of medical visual navigation according to the concept of the invention, building on the system in FIG. 2;

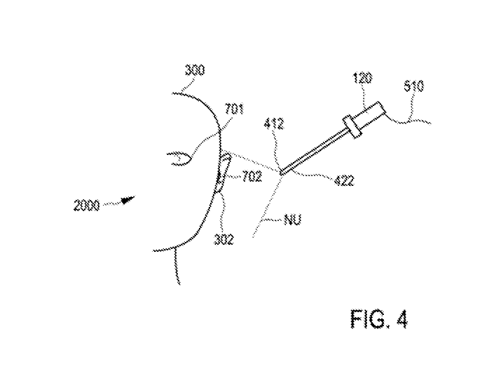

FIG. 4 shows an application for the purpose of implementing a patient registration method by means of a mobile maneuverable device as shown in FIG. 1 (B);

FIG. 5 shows a principle sketch for the purpose of explaining the SLAM method, wherein a so-called feature point matching is used in order to estimate a movement state of an object—e.g. the device head;

FIG. 6 shows a further preferred embodiment for the purpose of processing images taken at different times, in a mobile maneuverable device;

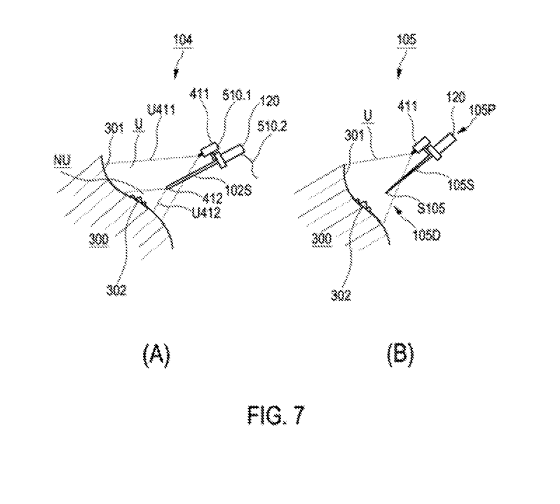

FIG. 7 shows yet another preferred embodiment of a mobile maneuverable device having a mobile device head, in view (A) with an internal and external camera, and in view (B) only with an external camera in the form of an endoscope and/or a pointer instrument;

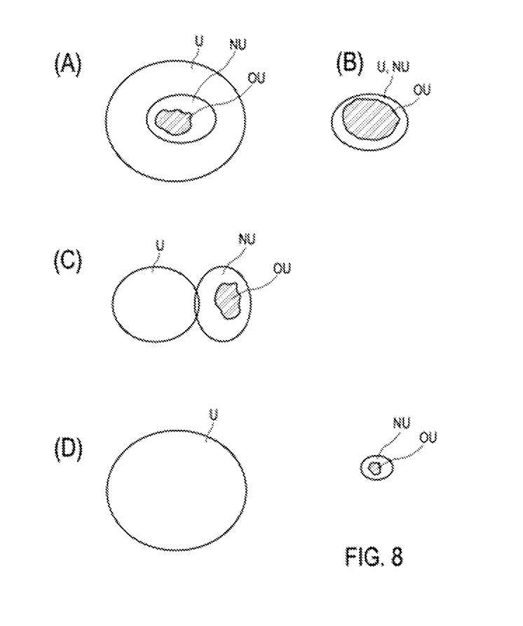

FIG. 8 shows a schematic illustration of different constellations, realized by one or more cameras, of a near environment which includes an operation environment, as well as an environment, wherein in particular the first [near environment] is visualized, and serves the purpose of an intervention in a body tissue, or generally a body, and wherein the latter [environment] particularly primarily serves the purpose of mapping and navigation, but without visualization;

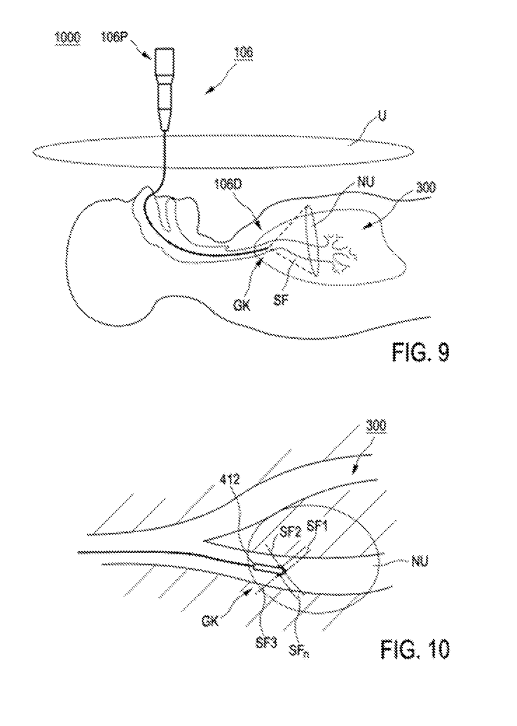

FIG. 9 shows an illustration for one example of a preferred embodiment; and

FIG. 10 shows a detail of the illustration for the example in FIG. 9.

The same reference numbers are used throughout the figure descriptions, with reference to the corresponding description portions, for identical or similar features, or features with identical or similar functions.

FIG. 1 shows, by way of example, as part of a mobile maneuverable device 1000 which is described in greater detail in FIG. 2 and FIG. 3, a mobile device head 101 which is designed for manual or automatic guidance, shown in reference to a body 300. The body 300 has an application region 301, wherein the mobile device head 101 is intended to be moved into proximity with the same—and this for the purpose of working on or observing the application region 301. In the present case, the body is constituted, as part of a medical application, by a tissue of a human or animal body, and has a depression 302 in the application region 301, which in the present case means a region which is free of tissue. The device head 101 in the present case is an instrument configured with a pincer or gripping device on the distal end 101D—indicated as the instrument head 110—and with a maneuvering device attached to the proximal end 101P, said maneuvering device not illustrated in view (A) in greater detail, such as a grip (view (B)) or a rotor arm [sic: robot arm] (view (C)).

The device head therefore has an instrument head 110 on the distal end 101D, as a tool, which can be constructed as a pincer or gripper, but also as another tool head such as a grinder, scissors, a machining laser, or the like. The tool has a shaft 101S which extends between the distal end 101D and the proximal end 101P. In addition, the device head 101 has, to form a guide device 400 designed for the purpose of navigation, an image data capture device 410 and a movement module 421 in the form of a system of sensors—in this case an acceleration sensor or gyroscope. The image data capture device 410 and the movement module 420 in the present case are connected via a data cable 510 to further units of the guide device 400 for the purpose of transmitting image data and movement data. The image data capture device comprises, in the example shown in FIG. 1 (view (A)), an external, 2D or 3D camera fixed on the shaft 101S, while the mobile device head 101—regardless of whether inside or outside of the body 300—is moved, [sic] the installed camera continuously captures images. The movement data of the movement module 420 is likewise continuously supplied, and can be used the precision [sic] of the subsequent analysis of the data transmitted by means of the data cable 510.

View (B) in FIG. 1 shows a further embodiment of a mobile device head 102, having a distal end 102D and a proximal end 102P. A lens of an image data capture device 412, and of a movement module 422, are installed on the distal end 102D. The mobile device head 102 is therefore configured with an integrated 2D or 3D camera. On the proximal end 102P, the device head has a grip 120 where an operator 201—for example a doctor—can grip the instrument, in the form of an endoscope, and guide the same. The distal end 102D is then configured with an internal image data capture device 412, and a data cable 510 is guided in the shaft 102S to the proximal end 102P, and connects the device head 102 to further units of the guide device 400, the same explained in greater detail in FIG. 2 and FIG. 3, in a manner allowing data communication.

View (C) in FIG. 1 substantially shows the same situation as view (B)—however, in this case, for an automatically guided mobile device head 103 in the form of an endoscope. A maneuvering apparatus in the form of a robot 202, having a robot arm, is included in the present case, holding the mobile device head 103. The data cable 510 is guided along the robot arm.

FIG. 2 shows a mobile maneuverable device 1000 in a generalized form, having a device head 100, by way of example a mobile device head, which is designed for manual or automatic guidance, such as one of the device heads 101, 102, 103 shown in FIG. 1, by way of example. In order to make possible a manual or automatic guidance of the device head 100, a guide device 400 is included. The device head 100 can be guided by means of a maneuvering apparatus 200, for example [by] an operator 201 or a robot 202. In the case of an automatic guidance in FIG. 2, the maneuvering apparatus 200 is controlled via a controller 500.

The guide device used for navigation specifically has, in the device head 100, an image data capture device 410 and a movement module 420. In addition, the guide device has an image data processing device 430 and a navigation device 440, positioned outside of the device head 100, both of which are described in greater detail in reference to FIG. 3 below.

In addition, the guide device can optionally, but not necessarily, have an external image data capture device 450 and an external tracker 460. The external image data capture device is used, referring to FIG. 3 and FIG. 4, particularly in the pre-operative stage in order to supply an auxiliary image—for example based on CT or MRT—which can be utilized initially, or irregularly, for the purpose of complementing the image data capture device 430.

The image data capture device 410 is designed to particularly continuously capture and provide image data of a near environment of the device head 100. The image data is then made available to a navigation device 440 which is designed to generate a pose and/or movement 480 of the device head, by means of the image data and an image data stream, using a map 470 which is compiled by the image data capture device.

The functionality of the mobile maneuverable device 1000 is therefore as follows. Image data of the image data capture device 410 are supplied to the image data capture device [sic: image data processing device] 430 via an image data connection 511—for example a data cable 510. The data cable 510 transmits a camera signal of the camera.

Movement data of the movement module 420 is supplied to the navigation device 440 via a movement data connection 512—for example by means of the data cable 510. The image data capture device is designed to capture image data of a near environment of the device head 100 and provide the same for further processing. In particular, in the present case, the image data is continuously captured and provided [by] the image data capture device 410. The image data processing device 430 has a module 431 for the purpose of mapping the image data, particularly for the purpose of compiling a map of the near environment by means of the image data. The map 470 serves as a template for a navigation device 440 which is designed to indicate a pose (position and/or orientation) and/or movement of the device head 100 by means of the image data and an image data stream. The map 470 can be given, together with the pose and/or the movement 480 of the device head 100, to a controller 500. The controller 500 is designed to control a maneuvering apparatus 200 according to a pose and/or movement of the device head 100 and using the map, said maneuvering apparatus guiding the device head 100. For this purpose, the maneuvering apparatus 200 is connected to the controller 500 via a control connection 510. The device head 100 is coupled to the maneuvering apparatus via a data coupling 210 for the purpose of navigation of the device head 100.

The navigation device 440 has a suitable module 441 for the purpose of navigation, meaning particularly the analysis of a pose and/or movement of the device head 100 relative to the map.

Even if the units 430, 440, in this case with the modules 431, 441, are illustrated as individual components, it is nevertheless clear that these can also [be] distributed over the entire device 1000 as a multitude of components, and particularly can work together in combination.

If multiple device heads—such as 302 [sic] instruments, tools, or sensors, particularly an endoscope, a pointer instrument, or a surgical instruments [sic] are each used with at least one mounted imaging camera, it is then possible for all of these to access and/or update the same image map for the purpose of navigation.

By way of example, in the present case, a method is named for the purpose of the compilation of the map 470 and the navigation—that is, for the purpose of generating a pose and/or movement 480 in the map 470—which is also known as a simultaneous localization and mapping method (SLAM). The SLAM algorithm of the module 431 is together [sic] with an extended Kalman filter EKF in the present case, which is conducive to a real-time analysis for the navigation. The navigation is therefore undertaken by a movement recognition analysis based on the image data, and used for the position analysis (navigation). While the device head 100 is therefore moved outside or inside of a body 300 (FIG. 1A and/or FIG. 1B, C), the image data capture device 410 continuously captures images. The simultaneously applied SLAM method determines the movement of the camera relative to the environment, based on the image data, and compiles a map 470, which in this case is a 3D map in the form of a series of points, or in the form of a surface model, by means of the images, from different positions and orientations; the latter method, taking into account various different positions and orientations, is also called a 6D method, and particularly a 6D SLAM method. If a map of the application region 301 is already available, the map is either updated or used for navigation on this map 470, 480.

Following the concept of the invention, the movement sensor system, indicated in the present case as a movement module 420, such as acceleration and gyroscopic sensors, can significantly increase the precision of the map 470 in and of itself, as well as the precision of the navigation 480. At the same time, the concept is designed in such a manner that the calculation time which must be invested is sufficient for a real-time implementation. The data processing calculates the movement direction in space from captures at different time points. These data are, by way of example, redundantly compared with the data of the combined, further movement sensor system, particularly the acceleration and gyroscopic sensors. It can be contemplated that the data of the acceleration sensor are taken into account in the data processing of the captures. In this case, both sensor values complement each other, and the movement of the instrument can be calculated more precisely.

So that it is possible to navigate in the target region with image map support, an image map of the target region should first be compiled. This primarily occurs using the map 470 and pose or navigation 480, by the movement of the instrument, including the camera, along the entire, or in parts of, the target region—that is, essentially only using the image data.

Secondarily, there is also the possibility of compiling the image map at the beginning by external, mobile or stationary camera systems such as the external image data capture device 450, or to continuously update the image map. In particular, an initial or other manner of image map compilation can be advantageous. It is also possible to use the external image data of an external image data source or image data capture device 450 in order to visually detect the instrument or parts of the instrument. By way of example, it is possible to generate image maps using pre-operative image sources such as, by way of example, CT, DVT, or MRT, or intraoperative 3D image data of the patient.

In addition, a parallel usage of classical tracking methods—likewise secondarily—can be advantageous, in each case limited temporarily. Because the navigation 480 using the image map 470 is a “chicken and the egg” problem in which it is only possible to determine relative positions, the absolute position can only be estimated without a further method. The concept of the invention provides a flexible, precise, and real-time-capable solution approach to this problem. As a complement, in one implementation, the absolute position can be determined by means of known navigation methods—such as optical tracking, by way of example, in a tracker module 460. In this case, the determination of the absolute position is only necessary initially, or at regular intervals, such that this system of sensors is only used temporarily during the navigated application. By way of example, the optical connection is therefore no longer permanently necessary between [the] markers and [the] optical tracking camera. As soon as the relative position between the camera and/or camera image data and the tracking system used is better known, the calculated map data of the surfaces can also be used for the image data recording.

The modules 450, 460, however, are fundamentally optional. In the device illustrated at present, the use of additional modules, such as an external image data source 450—particularly external images from CT, MRT, or the like—and/or external tracker modules 460 is only utilized to a limited degree, and/or the device is utilized entirely without the same. In particular, the presently described device 1000 therefore works without classical navigation sensors such as optical or electromagnetic tracking.

As concerns the navigation 480 and the compiling of the map 470 and the control 500 of the maneuvering apparatus 200, this is performed to a sufficient degree primarily, particularly as the sole significant approach, using the image data for the purpose of compiling the map 470 and for the purpose of navigation 480 on the map 470. The method and/or the device described in FIG. 2 can particularly, as explained by way of example with reference to FIG. 1, be used with respect to a tool, instrument, or a sensor for navigating the device, without classical measurement systems.

Because of the image- and/or map-support navigation, typical tracking methods are no longer necessary. In particular, in the case of the endoscope navigation, it is possible to use the integrated endoscope camera data (FIG. 1B, C). In addition, medical tools, by way of example, can be equipped with cameras (FIG. 1A) in order to navigate on the basis of the obtained images of the instrument, and optionally to compile a map. In the best case, even the endoscope can be excluded for the imaging.

In addition, a position and image data acquisition of the surfaces of a body can be carried out. It is possible to generate an intraoperative patient model, consisting of data of the surface including texturing of the operation region.

The method and the device 1000 serve the purpose of avoiding collisions, such that the compiled map 470 can also be used for the guiding of the device head 100, with no collisions, by means of a robot arm 202 or a similar automatic guidance, or by means of the maneuvering apparatus 200. It is possible for a doctor and/or user to avoid collisions, etc., or at least to be receive notification thereof, by the feedback mechanism or such a control loop, as described in FIG. 2 by way of example. In a combination of the automatic and manual guidance—for example FIGS. 1C and 1B—it is also possible to realize a semi-automatic operating mode.

An MCR module 432 has also proven advantageous, for example in the image data processing device 430, for the purpose of registering a movement of surfaces and for compensating movement (MCR: motion clutter removal). The continuous capture of image data of the same region by the endoscope can be falsified by a movement of the same surface, for example by breathing and heart beats. Because many organic movements can be described with harmonic, even, and/or repeating movements, the image processing can recognize such movements. The navigation can accordingly be matched. The doctor is informed of these movements visually and/or by feedback. It is possible to calculate, indicate, and use a prediction of the movement.

The device can be optimally expanded for automatic 3D image registering, as is described by way of example with reference to FIG. 3 and FIG. 4. By means of image registering methods and/or 3D matching algorithms for the purpose of recognizing identical 3D data and/or surfaces from different imaging methods, it is possible in the instrument navigation 480 presented to connect the 3D map 470 with volume data sets of the patient. These can be CT or MRT datasets. As such, the surface and the underlying tissue and structures are known to the doctor. In addition, this data can be taken into account for the operation planning.

Specifically, FIG. 3 shows the basic concept of the medical, visual navigation presented here with respect to the example in FIG. 1B. Again, identical reference numbers are used for identical or similar features or features having identical or similar functions. The image data capture device 412 in the form of a camera supplies image data of a near environment U, particularly the capture region of the camera. The image data relate to a surface of the application region 301. The data are saved as image B301 in an image map memory, as an image map 470. The map 470 can also be saved in another memory. As such, the map memory constitutes the image map 470 saved so far.

A structure 302 below the surface can be saved as image B302 in a preoperative source 450 as a CT, MRT, or similar image. The preoperative source 450 can comprise a 3D image data memory. As such, the preoperative source constitutes 3D image data of the near environment U and/or the underlying structures. The map 470 is combined with the data of the preoperative source 450 by means of the image data processing device and the navigation device 430, 440, to give a visual synopsis of the map 470 and navigation information 480 on the mobile device head—in this case in the form of the endoscope—and/or the determination of the pose and movement in the capture region of the camera—meaning the near environment U. The output can be done on a visual capture device 600 illustrated in FIG. 2. The visual image data capture device 600 can include an output device for the position-overlapped representation of image data and current instrument positions.

The synopsis of the images B301 and B302 is a combination of current surface maps of the instrument camera and the 3D image data of the preoperative source. The connection 471 between the image- and data processing device and the image map memory also comprises a connection between the image data processing device and the navigation device 430, 440. These comprise the SLAM and EKF modules explained above.

The current detected position of the instrument is also called “matching” the instrument. Other image aspects can also be matched—for example a band of prominent points. FIG. 4 shows, as an example, a preferred arrangement of the mobile device in FIG. 1(B) for the purpose of registering a patient 2000, wherein an overlapping with external image data as described above is also provided. By way of example, in an application region 301, 302 of a body 300 of the patient 2000, the surfaces of eyes, noses, ears, or teeth can be used for the patient registration. External image data (e.g. CT data of the area) can be automatically or manually combined with the image map data [of this method] of the near environment, [which] substantially corresponds to the capture region of the camera. The automatic method can be realized with 3D matching methods, by way of example.

A manual overlapping of external image data with the image map data can be performed, by way of example, by the user marking a series of prominent points 701, 702 (for example, the subnasal [point] and corner of the eye) in both the CT data and in the map data.

FIG. 5 schematically shows the principle of the SLAM method for simultaneous localization and mapping. This is performed in the present case using so-called feature point matching with prominent points (e.g. 701, 702 in FIG. 4 or other prominent points 703, 704, 705, 706), and an estimation of the movement. However, the SLAM method is only one possible option for implementing the concept of the invention explained above. The method exclusively uses the sensor signals for orientation in an expanded region which is composed of a number of near environments. In this case, the movement [of the device] is estimated using the sensor data (typically image data BU), and a map 470.1, 470.2 of the detected region is continuously compiled. In addition to the compiling of the map, and the recognition of movement, the currently detected sensor information is simultaneously checked for agreement with the image map data saved so far. If an agreement is determined, then the system knows its own current position and orientation inside of the map. It is possible on this basis to specify comparably robust algorithms and successfully use the same. The “monocular SLAM” method has been presented for using 2D camera images as the information source. In this case, feature points 701, 702, 703, 704, 705, 706, of an object 700 are continuously detected in the video image, and the movement thereof is analyzed in the image. FIG. 5 shows the feature points 701, 702, 703, 704, 705, 706 of an object 700 in view (A), and a movement of the same in view (B), toward the right rear (701′, 702′, 703′, 704′, 705′, 706′) of an object 700, wherein the length of the vector to the shifted object 700′ is a measure of the movement, particularly distance and speed.

FIG. 5 therefore specifically shows two images of a near environment BU, BU′ at a first capture time T1 and a second capture time T2. The prominent points 701 to 706 are functionally assigned to the first capture point T1, and the prominent points 701′ to 706′ are functionally assigned to the second capture point T2. This means that object 700 at time T1 appears at time T2 as object 700′ with a different object position and/or orientation. The vectors, which are not drawn in greater detail, between the prominent points (that is, the vectors between points 701, 701′ and 702, 702′, and 703, 703′, and 704, 704′, and 705, 705′, and 706, 706′) which are functionally assigned to the time point[s]—by way of example vector V—indicate the distance, and, via the time difference between the time points T1 and T2, the speed of the relationship between objects 700 and 700′.

In the form just shown in FIG. 5, it can therefore be seen that the object 700 at time T1 has been clearly shifted back and to the right at a speed which can be determined from the time points T1 and T2. It is accordingly possible to determine therefrom the movement of an image data capture device 410, particularly a lens, on the distal end 101D or 102D of a device head.

FIG. 6 shows how, by means of this method, it is possible to combine camera images (the endoscope camera in this case) into a map, and to illustrate the camera images as a patient model in a shared 3D view.

In this regard, FIG. 6 shows a mobile maneuverable device 1000 as has been explained fundamentally with reference to FIG. 2 and FIG. 3, wherein again the same reference numbers are used for identical or similar parts or parts having identical or similar functions, such that reference concerning the same is hereby made to the description of FIG. 2 and FIG. 3 above. FIG. 6 shows the device with a mobile device head 300 at three different time points T1, T2, T3—particularly the mobile device heads 100T1, 100T2, and 100T3 shifted in time. The near environment U of the mobile device head 100, determined substantially by means of an image data capture device 410 using a capture region of a camera or the like, is capable of leaving a certain region 303 of the body 300, said region being mapped, by the device head 100 being moved and assuming different positions at the time points T1, T2, T3. The region 303 being mapped therefore is composed of a capture region of the near environment U1 at time point T1 and a capture region at time point T2 corresponding to the near environment U2 and a capture region of the near environment U3 at time point T3. Corresponding image data transmitted to the visual image data capture device 600 or a similar monitor via the data cable 510 represents the region being mapped as image B303. As such, the same is composed of a sequence of images of which three images BU1, BU2, BU3 are shown, the same corresponding to the time points T1, T2, T3. By way of example, this could be an image B301 of the application region 301 or the depression 302 in FIG. 1, or another image representation of the structure 310. The surface of the body 300 can fundamentally be reproduced in the form of the structure 310 in the region 303 being mapped as image B303—that is, the surface which can be captured by a camera. What can be captured in this case is not necessarily limited to the surface. Rather, it can partially penetrate to a depth depending on the characteristic of the image data capture device—specifically the camera.

In principle, the camera installed in the endoscope, particularly in the case of an endoscope, can be used as the camera system. In the case of 2D cameras, the 3D image information can be calculated and/or estimated from image sequences and a movement of the camera. In particular, in the case of instruments, cameras can also be contemplated at other positions of the instrument and/or endoscope—such as on the shaft, by way of example. All known types of cameras can be considered as the camera—particularly unidirectional and omnidirectional 2D cameras or 3D camera systems, for example with stereoscopy or time of flight methods. In addition, 3D image data can be calculated using multiple 2D cameras installed on the instrument, or the quality of the image data can be improved using multiple 2D and 3D cameras. Camera systems detect, in the most common cases, light of visible wavelengths between 400 and 800 nanometers. However, further wavelength regions, such as infrared or UV, can also be used in the use with these systems. The use of further sensor systems can also be contemplated.

Image data acquisition, such as radar or ultrasound systems, for example, for capturing the surface, or optionally deeper, reflecting or emitting layers [sic]. Particularly to detect rapid movements of the instrument, camera systems having a particularly high image capture frequency, up to high-speed cameras, are particularly advantageous.

FIG. 7 shows examples of preferred possibilities for a further external camera position on an instrument. Because the region of the image data used for navigation is fundamentally insignificant, a camera can also be mounted at further positions on the instrument, such that the movement of the endoscope and the assignment of the position is still possible, or is more precise.

FIG. 7 shows a further example of a device head 104 in view (A), in the form of an endoscope, wherein the same reference numbers are used for identical or similar parts and/or parts having identical or similar functions, as in FIG. 1B and FIG. 1C. The device head in the present case has a first image data capture device 411 in the form of an external camera attached on the shaft 102S or on the grip 120 of the endoscope, and a second image data capture device 412 integrated into the interior of the endoscope in the form of a further camera—particularly the endoscope camera. The external camera 411 has a first capture region U411 and the internal camera has a second capture region U412. The image data captured in the first capture region U411, and/or a first near environment determined by the same, is transmitted via a first data cable 510.1 to a guide device 400. Image data of a second capture region U412 and/or a second near environment determined thereby is likewise transmitted to the guide device 400 by a second data cable 510.2 of the endoscope. As concerns the guide device 400, reference is hereby made to the description of FIG. 2 and FIG. 3, wherein the image data connection 511 created via the data cable is shown, for the connection of the image data capture device 410 and an image data processing device and/or navigation device 430, 440. Accordingly, the image data capture device 410 illustrated in FIG. 2 can have two image data capture devices as illustrated as an example in FIG. 7A, for example the image data capture devices 411, 412 as illustrated in FIG. 7A.

The availability of two images at the same time of a first and a second near environment, with a capture region which partially overlaps in each case, from different perspectives, can be used in an image data processing device and/or the navigation device 430, 440 via computation for the purpose of improving the precision.

The system is also functional if the camera never penetrates into the body. Of course, to increase the precision, multiple cameras can be operated on an instrument at the same time. Moreover, it can be contemplated that instruments and pointer instruments are used together with an installed camera. By way of example, if the relative position of the tip of the pointer instrument with respect to the camera and/or to the 3D image data is known, it is possible to carry out a patient registration by means of this pointer instrument, or an instrument which can be used similarly.

In this regard, FIG. 7(B) shows a further embodiment of a mobile device head 105 in the form of a pointer instrument, wherein again the same reference numbers are used for identical or similar parts and/or parts having identical or similar functions, as in the figures above. The pointer instrument has a pointer tip S105 on the distal end 105D of the shaft 1055 of the pointer instrument 105. The pointer instrument also has a grip 120 on the proximal end 105P. In the present case, an image data capture device 411 is attached on the grip 120 as the only camera of the pointer instrument. For the determination of the near environment, the tip S105 and/or the distal end 105D of the pointer instrument 105, as well as the application region 301, are substantially in the capture region of the image data capture device 411. As such, it is possible to capture and map a structure 302 which the tip S105 of the pointer instrument 105 faces, by means of the camera, together with the relative position of the tip S105 and the structure 302—that is, a pose of the tip 105 relative to the structure 302.

In FIG. 7(A), the capture regions U411, U412 of the first and second camera 411, 412 overlap in such a manner that the structure 302 lies in the overlap region.

It should be understood that a guiding means which has a position reference to the device head and is functionally assigned to the same is designed to give details on the position of the device head 100 with respect to the environment U in the map 470, wherein the environment U extends beyond the near environment NU can be [sic] included alone to compile a map. This is the case in FIG. 7(B), for example. Nevertheless, it is particularly preferred that guiding means are included additionally to an image data capture device 412, e.g., if the latter is installed in the device head.

In one modification, an image data capture device 412 can also be employed in two roles, such that the same serves the purpose of mapping an environment and also visually capturing a near environment. This can be the case, by way of example, if the near environment is an operation environment of the distal end of the mobile device head 100—for example with a lesion. The near environment NU can then further comprise the image data which is captured in the visual range of a first lens 412 of the image data capture device 410 on the distal end of the mobile device head 100. The environment U can include a region which lies in the near environment NU and beyond the operation environment of the distal end of the mobile device head 100.

Image capture devices (such as the cameras 411, 412 in FIG. 7(A), for example) can fundamentally be installed at different, and any arbitrary, positions on the instrument, and in this case in the same or different directions, in order in the latter case to be able to capture different near and (distant) environments.

A near environment in this case commonly includes an operation environment of the distal end of the mobile device head 100 into which the operator reaches. The operation region and/or the near environment is, however, not necessarily the region being mapped. In particular, following the example in FIG. 7(B), it is possible that the near environment is not visualized and/or captured directly proximate to the distal end of the mobile device head 100 (e.g. if only a pointer or a surgical instrument is used in place of the endoscope). In this case, as explained above in reference to FIG. 7(B), the environment U can extend beyond the near environment NU and be included solely for the purpose of compiling a map.

FIG. 8 shows, in view (A), an arrangement of an environment U which is representative, among other things, for the situation in FIG. 7(A), with a near environment NU arranged entirely inside the same, both of which are functionally assigned to a field of vision of an internal camera 412 and/or external camera 411. The shaded region of the near environment in this case serves as an operation environment OU for an intervention into a body tissue. The entire region of the environment U serves the purpose of mapping, and therefore of navigation of an instrument, such as the internal sight camera 412 on the distal end of the endoscope in this case.

FIG. 8(A) also illustrates, in a modified form, an example according to FIG. 1(A), wherein an environment U serves the purpose of mapping, an operation environment OU [sic], but is not visualized (to the extent that a near environment NU is not present), because no internal camera is attached on the distal end of the device head. Rather, in this case, only a surgical instrument head is attached, in the example in FIG. 1(A).

FIG. 8(B) shows that the regions of an environment U, a near environment NU, and the operation environment OU can also more or less coincide with each other. This can particularly be the case in an example in FIG. 1(B) or FIG. 1(C). In this case, an internal sight camera 412 of the endoscope is particularly used to monitor tissue in an operation environment OU in the region of the near environment NU (that is, in the field of vision of the internal camera 412). The same region also serves, as the environment U, the purpose of mapping, and therefore of navigation of the distal end 101D of the endoscope.

FIG. 8(C) illustrates a situation already described above in which the near environment NU and the environment U lie next to each other, and touch each other or partially overlap, wherein the environment U serves the purpose of mapping, and only the near environment NU comprises the operation environment OU. This can arise, by way of example, for cartilage or bone regions in the environment U, and a mucous membrane region in the near environment NU, wherein the mucous membrane simultaneously comprises the operation environment. In this case, the mucous membrane only provides poor starting points for mapping because it is comparably diffuse, while a cartilage or bone in the environment U has sight positions which can serve as markers, and can therefore be the basis for a navigation.

The same can be true for the example in FIG. 8(A) explained above, wherein an environment U of solid tissue such as cartilage or bone is present in a region arranged approximately in a ring, said tissue being well suited for mapping, while blood or nerve vessels are arranged in a region of a near environment NU lying therein.

As shown in FIG. 8(D), however, the situation can also be such that an environment U and a near environment NU are disjunct—that is they constitute image regions which are localized completely independently of each other. In an extreme case, but particularly preferred, an environment U can lie in the field of vision of an external camera, by way of example, and can comprise operation devices, an operating room, or orientation objects in a space which is significantly beyond the near environment NU. This can also, in a less extreme case, be the environment U on the surface of a face of a patient. The face often is suitable for providing marker positions, as a result of prominent points such as a pupil of an eye or a nose opening, by means of which a comparably good navigation is possible. The operation region in the near environment NU can deviate therefrom significantly—for example including a nasal cavity or a region in the throat of a patient and/or below the surface of the face, i.e. in the interior of the head.

### EXAMPLE

FIG. 9 shows one example of an application of a mobile maneuverable device 1000, having a mobile device head 106 in the form of a moveable endoscope and/or bronchoscope, potentially also with instruments such as a biopsy needle on the device head GK, for example. As such, a bronchoscope or endoscope used in the operating room, with a camera module or with a miniaturized camera module on the distal end 106D—as shown approximately in FIG. 10—with a flexible holder on a proximal end 106P, can serve as hardware. The pose in the local map (map of the near environment NU) is known as a result of successive reconstruction of the environment map (map of the environment U) and estimation of the position and orientation (pose) of the object in the environment map. A global map—that is, corresponding to the environment map, or as a map of the environment U which complements the same, or as part of the same—can be compiled by the surface model from a 3D dataset (by way of example CT (computer tomography) or MRT (magnetic resonance tomography)) captured most commonly prior to the operation. The local map of the near environment NU is registered to the global map of the environment U, thereby giving an objective position in the global map. In addition—similarly to the principle of augmented reality—the path to the target region which has been marked in the 3D dataset can be displayed in the camera image for the operator. One advantage lies in the possibility of navigating inside the human body using flexible, bendable medical instruments or other device heads—such as a device head 106 in this case having an endoscope and/or bronchoscope head as the device head GK, optionally with a biopsy needle on the distal end 106D. Local post determination is possible in the navigation independently of soft tissue partial movements—due for example to the breathing of the patient. The local deformation of the bronchia is only very minimal, but the absolute deviation of the position is significant. On the basis of the concept of the invention described herein, a position detection of a device head GK on the distal end 106D of the device head 106 is made possible even in structures of soft tissue, and simplifies the position determination of these structures in datasets captured preoperatively.

FIG. 10 shows a camera characteristic for the purpose of illustrating an image data capture device 412 on the device head GK of the device head 106 on the distal end 106D of the same, in the case of a moveable instrument—in this case an endoscope or bronchoscope in FIG. 9. It is possible to form an expanded field of vision SF for the purpose of portraying a near environment NU using fields of vision SF1, SF2, SF3 . . . SFn of multiple cameras, or to provide a camera with a further field of vision SF for the purpose of portraying a near environment NU. Camera heads with image capture and illumination in multiple directions for the fields of vision SF1, SF2, SF3 . . . SFn and/or for a wide field of vision SF are advantageous.

List of reference numbers

| B301, B302, B303 | images |
|---|---|
| BU | image data |
| EKF | extended Kalman filter |
| GK | device head |
| S105 | tip |
| T1 | first timepoint |
| T2 | second timepoint |
| T3 | third timepoint |
| U, U1, U2, U3 | environment |
| NU | near environment |
| SF, SF1, SF2, SF3, SFₙ | field of vision |
| U411, U412 | capture region |
| V | vector |
| 100, 100T1, 100T2, 100T3 | device head |
| 101, 102, 103, 104, 105, 106 | mobile device head |
| 101D, 102D, 105D, 106D | distal end |
| 101P, 102P, 105P, 106P | proximal end |
| 101S, 102S, 105S | shaft |
| 110 | instrument head |
| 120 | grip |
| 200 | maneuvering apparatus |
| 201 | operator |
| 202 | robot, robot arm |
| 210 | data coupling |
| 300 | body |
| 301 | application region |
| 302 | depression, structure |
| 303 | mapping region |
| 400 | guide device |
| 410, 411, 412 | image data capture device |
| 420, 421, 422 | movement module |
| 430 | image data processing device |
| 431, 441 | module |
| 432 | MCR module (motion clutter removal) |
| 440 | navigation device |
| 450, 460 | tracker module |
| 450 | external image data source, |
| | preoperative source |
| 470, 470.1, 470.2 | map, image map |
| 471 | connection |
| 480 | pose and/or movement |
| 500 | controller |
| 510, 510.1, 510.2 | data cable |
| 511 | image data connection |
| 512 | movement data connection |
| 600 | visual capture device |
| 700 | object |
| 701, 702, 703, 704, 705, 706 | prominent points (feature points) |
| 701′, 702′, 703′, | prominent points (feature points) |
| 704′, 705′, 706′ | |
| 1000 | mobile maneuverable device |
| 2000 | patient |

1. A mobile, maneuverable, particularly calibratable device having a mobile device head, particularly a non-medical mobile device head with a distal end for arrangement relative to a technical body, or a medical, mobile device head having a distal end for arrangement relative to a tissue-like body, particularly having a distal end for insertion or attachment on the body, having:
at least one mobile device head for manual or automatic guidance,
a guide device, wherein the guide device is designed for the purpose of providing navigation information for the guidance of the mobile device head wherein the distal end thereof can be guided in a near environment,
an image data capture device which is designed to capture image data of an environment of the device head, particularly continuously, and to make the same available,
an image data processing device which is designed for the purpose of compiling a map of the environment, and
a navigation device which is designed to indicate at least one position of the device head in the near environment using the map, by means of the image data and an image data stream, in such a manner that the mobile device head can be guided using the map, wherein
a guiding means, with a position reference to the device head, functionally assigned to the same, which is designed to give specifications on the position of the device head in the map with respect to the environment, wherein the environment extends beyond the near environment.

2. A device according to claim 1, characterized in that the position reference of the guiding means to the device head is stationary or moveable in a deterministic way, and particularly can be calibrated.

3. A device according to claim 1, characterized in that the guiding means comprises the image data capture device and/or a further orientation module which is designed to provide a further specification on the position, particularly the pose (position and orientation) and/or movement of the device head with respect to the map.

4. A device according to claim 1, characterized in that the orientation module comprises a movement module and/or acceleration sensor or similar system of sensors, and/or the orientation module comprises at least one lens particularly a target sighting lens and/or guide lens and/or an external lens.

5. A device according to claim 1, characterized in that it is possible to specify a position, particularly a pose and/or movement of the device head in the near environment using the map, in such a manner that a controller and a maneuvering apparatus can guide the mobile device head according to the position, particularly the position and/or orientation and/or movement of the device head, and using the map of the environment.

6. A device according to claim 1, characterized in that the maneuvering apparatus is designed to automatically guide the mobile device head by means of the controller, via a control connection, and the controller is designed for the purpose of navigation of the device head, by means of the guide device, via a data coupling, and particularly the control connection is designed for the purpose of transmitting a TARGET position, particularly a pose, and/or a TARGET movement of the device head, and the data coupling is designed for the transmission of a CURRENT position, particularly a pose, and/or a CURRENT movement of the device head.

7. A device according to claim 1, characterized in that the navigation device has an extended Kalman filter and/or a module for executing a SLAM algorithm.

8. A device according to claim 1, characterized in that the guide device is further designed to guide the at least one mobile device head only using the map, and particularly the guide device has an absolute tracking module, particularly a further sensor system, which can be temporarily activated with limited functionality, or deactivated, and/or can be partially activated or deactivated for the purpose of compiling the map of the near environment.

9. A device according to claim 1, characterized in that the device head is a first mobile device head, and it is possible to guide at least one second mobile device head, particularly a plurality of mobile device heads, using the map, particularly the same, single map.

10. A device according to claim 1, characterized in that the map contains markers, and the guiding means is designed to recognize at least one unknown or known body shape in the environment as a marker, wherein it is possible to determine the relative position, particularly the relative pose, of the device head to a position, particularly a position of the known body shape, and particularly it is possible to determine, simultaneously, a relative position, particularly a relative pose, of a number of body shapes with respect to each other and/or to the device head.

11. A device according to claim 1, characterized in that the map contains markers, wherein objects are inserted into the environment as markers, and these are particularly suited to being detected by the image data capture device, and particularly the guiding means is designed to measure a fixed object a single time, and for multiple uses as a marker, or to measure the same continuously.

12. A device according to claim 1, characterized in that the position, particularly the pose and/or movement, of the device head can be specified using the map relative to a reference point on a body shape or an object in the environment of the device head, wherein the reference point is part of the map of the environment, particularly the near environment.

13. A device according to claim 1, characterized in that a reference point lies outside of the near environment, wherein it is possible to specify a determined relation between the reference point and a map position.

14. A device according to claim 1, characterized in that the guiding means is designed to measure a moving mechanism or a movement kinematics for position deviations, one time, at regular intervals, or continuously.

15. A device according to claim 1, characterized in that the image data processing device is designed for the purpose of identifying a reference point on an object in a visual image, with a fixed point of an auxiliary image following a certain test.

16. A device according to claim 1, characterized in that the image data processing device is designed to register and/or add to a visual image, particularly the map, with an auxiliary image, particularly a computer tomography or magnetic resonance tomography image (CT or MRT image) or a similar image, particularly initially, at regular intervals, or continuously.

17. A device according to claim 1, characterized in that the image data processing device has a module which is designed to recognize target movements, particularly target body movements, particularly target body movements which can be recognized according to a physiological pattern and are particularly rhythmic, and to take the same into account for the compiling of a map of the near environment.

18. A device according to claim 1, characterized in that the environment includes the near environment.

19. A device according to claim 1, characterized in that the environment is disjunct from the near environment.

20. A device according to claim 1, characterized in that the near environment is an operation environment of the distal end of the mobile device head.

21. A device according to claim 1, characterized in that the near environment comprises the image data which is captured in the visual range of a first lens of the image data capture device on the distal end of the mobile device head.

22. A device according to claim 1, characterized in that the environment includes a region which lies in the near environment and beyond the operation environment of the distal end of the mobile device head.

23. A device according to claim 1, characterized in that at least one first and one second region of the environment can be captured by the guiding means and/or the image data capture device, wherein at least the first region is a part of an operation environment in which a movement can be detected, particularly a movement of a body shape and/or a distal end of the device head.

24. A device according to claim 1, characterized in that at least one first and one second region of the environment can be captured by the guiding means and/or the image data capture device, wherein in at least the first captured region it is possible to detect and/or compensate for, by means of the guiding means, errors, failures, or lost signals or similar malfunctions by means of the analysis of at least the second region.

25. A device according to claim 1, characterized in that the guiding means, particularly the image data capture device and/or the orientation module is/are designed to continuously capture the image data of the environment of the device head, and the image data processing device is designed to compile a map of the environment by means of the image data, particularly to specify further details on the position—particularly the pose and/or movement of the device head with respect to the map in real-time, particularly during an operation.

26. A device according to claim 1, characterized in that the image data capture device has at least one, particularly two, three or another number of lenses, which are designed to simultaneously capture the same region or further regions of the environment particularly regions in a near environment and/or in addition to the near environment by means of image data, and particularly a movement of an instrument which can move, can be determined using at least two lenses.

27. A device according to claim 1, characterized in that a first lens captures first image data and a second lens captures second image data, wherein the first and second image data are captured at the same time, and are offset spatially, or wherein the first and second image data are captured in the same space, and at different times.

28. A device according to claim 1, characterized in that the image data capture device has a sighting lens which sits on a distal end of the device head, wherein the sighting lens is designed to capture image data of a near environment on a distal end of the device head.

29. A device according to claim 1, characterized in that the guiding means, particularly the image data capture device and/or the orientation module, has a guide lens which sits on a guide point—at a distance from a distal end, particularly on a proximal end of the device head and/or on the guide device wherein the guide lens is designed to capture image data of an environment of the device head, particularly near to the guide point.

30. A method for the maneuvering, and particularly calibration, of a device having a mobile device head, particularly a non-medical mobile device head having a distal end for arrangement relative to a technical body, or a medical, mobile device head having a distal end for arrangement relative to a tissue-like body, particularly having a distal end for insertion or attachment on the body, having the steps:
manual or automatic guidance of the mobile device head,
provision of navigation information for the guidance of the mobile device head, wherein the distal end thereof is guided in a near environment,
capturing and provision of image data of an environment of the device head, particularly continuously,
compiling of a map of the environment by means of the image data,
specifying at least one position of the device head in the near environment using the map, by means of the image data and an image data stream, in such a manner that the mobile device head can be guided using the map, wherein
it is possible to provide details of the position of the device head in the map with respect to the environment, with the position reference to the device head, wherein the environment extends beyond the near environment.

31. A method according to claim 30, characterized in that the position reference to the device head is stationary or moves in a determined manner, and particularly is calibrated.

32. A method according to claim 30, characterized in that a further specification on the position, particularly the pose and/or the movement of the device head is provided in reference to the map.

33. A method according to claim 30, characterized in that the mobile device head is guided automatically, wherein a TARGET position, particularly pose, and/or a TARGET movement of the device head is transmitted, and a CURRENT position, particularly pose, and/or CURRENT movement of the device head is transmitted.

34. A device according to claim 2, characterized in that:
the guiding means comprises the image data capture device and/or a further orientation module which is designed to provide a further specification on the position, particularly the pose and/or movement of the device head with respect to the map;
the orientation module comprises a movement module and/or acceleration sensor or similar system of sensors, and/or the orientation module comprises at least one lens, particularly a target sighting lens and/or guide lens and/or an external lens;
it is possible to specify a position, particularly a pose and/or movement of the device head in the near environment using the map, in such a manner that a controller and a maneuvering apparatus can guide the mobile device head according to the position, particularly the position and/or orientation and/or movement of the device head, and using the map of the environment;
the maneuvering apparatus is designed to automatically guide the mobile device head by means of the controller, via a control connection, and the controller is designed for the purpose of navigation of the device head, by means of the guide device, via a data coupling, and particularly the control connection is designed for the purpose of transmitting a TARGET position, particularly a pose, and/or a TARGET movement of the device head, and the data coupling is designed for the transmission of a CURRENT position, particularly a pose, and/or a CURRENT movement of the device head;
the navigation device has an extended Kalman filter (EKF) and/or a module for executing a SLAM algorithm;
the guide device is further designed to guide the at least one mobile device head only using the map, and particularly the guide device has an absolute tracking module, particularly a further sensor system, which can be temporarily activated with limited functionality, or deactivated, and/or can be partially activated or deactivated for the purpose of compiling the map of the near environment;
the device head is a first mobile device head, and it is possible to guide at least one second mobile device head, particularly a plurality of mobile device heads, using the map, particularly the same, single map;
the map contains markers, and the guiding means is designed to recognize at least one unknown or known body shape in the environment as a marker, wherein it is possible to determine the relative position, particularly the relative pose, of the device head to a position, particularly a position of the known body shape, and particularly it is possible to determine, simultaneously, a relative position, particularly a relative pose, of a number of body shapes with respect to each other and/or to the device head;
the map contains markers, wherein objects are inserted into the environment as markers, and these are particularly suited to being detected by the image data capture device, and particularly the guiding means is designed to measure a fixed object a single time, and for multiple uses as a marker, or to measure the same continuously;
the position, particularly the pose and/or movement, of the device head can be specified using the map relative to a reference point on a body shape or an object in the environment of the device head, wherein the reference point is part of the map of the environment, particularly the near environment;
a reference point lies outside of the near environment, wherein it is possible to specify a determined relation between the reference point and a map position;
the guiding means is designed to measure a moving mechanism or a movement kinematics for position deviations, one time, at regular intervals, or continuously;
the image data processing device is designed for the purpose of identifying a reference point on an object in a visual image, with a fixed point of an auxiliary image following a certain test;
the image data processing device is designed to register and/or add to a visual image, particularly the map, with an auxiliary image, particularly a computer tomography or magnetic resonance tomography image (CT or MRT image) or a similar image, particularly initially, at regular intervals, or continuously;
the image data processing device has a module which is designed to recognize target movements, particularly target body movements, particularly target body movements which can be recognized according to a physiological pattern and are particularly rhythmic, and to take the same into account for the compiling of a map of the near environment;
the environment includes the near environment;
the environment is disjunct from the near environment;
the near environment is an operation environment of the distal end of the mobile device head;
the near environment comprises the image data which is captured in the visual range of a first lens of the image data capture device on the distal end of the mobile device head;
the environment includes a region which lies in the near environment and beyond the operation environment of the distal end of the mobile device head;
at least one first and one second region of the environment can be captured by the guiding means and/or the image data capture device, wherein at least the first region is a part of an operation environment in which a movement can be detected, particularly a movement of a body shape and/or a distal end of the device head;
at least one first and one second region of the environment can be captured by the guiding means and/or the image data capture device, wherein in at least the first captured region it is possible to detect and/or compensate for, by means of the guiding means, errors, failures, or lost signals or similar malfunctions by means of the analysis of at least the second region;
the guiding means, particularly the image data capture device and/or the orientation module is/are designed to continuously capture the image data of the environment of the device head, and the image data processing device is designed to compile a map of the environment by means of the image data, particularly to specify further details on the position—particularly the pose and/or movement of the device head with respect to the map—in real-time, particularly during an operation;
the image data capture device has at least one, particularly two, three or another number of lenses, which are designed to simultaneously capture the same region or further regions of the environment—particularly regions in a near environment and/or in addition to the near environment—by means of image data, and particularly a movement of an instrument which can move, can be determined using at least two lenses;
a first lens captures first image data and a second lens captures second image data, wherein the first and second image data are captured at the same time, and are offset spatially, or wherein the first and second image data are captured in the same space, and at different times;
the image data capture device has a sighting lens which sits on a distal end of the device head, wherein the sighting lens is designed to capture image data of a near environment on a distal end of the device head; and
the guiding means, particularly the image data capture device and/or the orientation module, has a guide lens which sits on a guide point—at a distance from a distal end, particularly on a proximal end of the device head and/or on the guide device—wherein the guide lens is designed to capture image data of an environment of the device head, particularly near to the guide point.

35. A method according to claim 31, characterized in that:
a further specification on the position, particularly the pose (position and/or orientation) and/or the movement of the device head is provided in reference to the map; and
the mobile device head is guided automatically, wherein a TARGET position, particularly pose, and/or a TARGET movement of the device head is transmitted, and a CURRENT position, particularly pose, and/or CURRENT movement of the device head is transmitted.
